(19) United States
(12) Patent Application Publication
(10) Pub. No.: US 2026/0191890 A1
(43) Pub. Date: Jul. 9, 2026

(54) Use of Morinda officinalis oligosaccharides and composition thereof in prevention or treatment of depression

(71) Applicant: NANJING HAIGUANG INSTITUTE OF APPLIED CHEMISTRY CO. LTD, Nanjing (CN)

(72) Inventors: Yan Wang, Haikou (CN); Xinjie Zhu, Haikou (CN); Haibin Luo, Haikou (CN); Song Li, Haikou (CN)

(21) Appl. No.: 18/839,390

(22) PCT Filed: Feb. 17, 2023
(86) PCT No.: PCT/CN2023/076694

§ 371 Date: Aug. 16, 2024

(30) Foreign Application Priority Data
Feb. 17, 2022 (CN) 202210146684.0
Feb. 17, 2022 (CN) 202210147289.4

(51) Int. Cl. A61K 31/702; A61K 36/746; A61P 25/24

(52) U.S. Cl. CPC A61K 31/702; A61K 36/746; A61P 25/24

(57) ABSTRACT

Use of Morinda officinalis oligosaccharides represented by the following structural formula and a composition comprising the same and an intestinal flora in the preparation of a medicine for the preventing or treating depression. By means of a Trp-5-HTP-5-HT pathway, Morinda officinalis oligosaccharides and the intestinal flora are combined for use so as to increase the content of 5-HTP in the blood and the content of 5-HTP and 5-HT in the brain, thereby improving the treatment on depression.

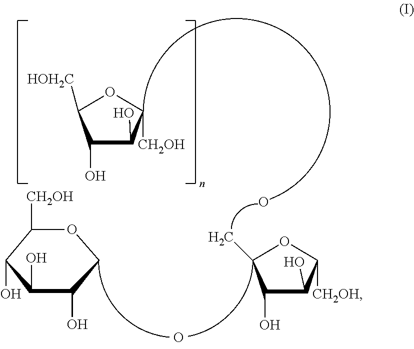

wherein n=1, 2, 3, 4, 5, 6 or 7.

The present application is based on the application with CN application number 202210146684.0 and application date Feb. 17, 2022, and the application with CN application number 202210147289.4 and application date Feb. 17, 2022, and claims the priority thereof. The disclosures of the CN applications are hereby incorporated by reference into the present application in their entirety.

### TECHNICAL FIELD

The present invention relates to the fields of bioengineering and medicine, in particular to the technical fields of traditional Chinese medicine and prevention and treatment of depression, and specifically to the use of Morinda officinalis oligosaccharides and composition thereof in the prevention or treatment of depression.

### BACKGROUND ART

Depression is a common emotional disorder that endangers human physical and mental health, characterized by significant and persistent low mood. Its main symptoms include depressed mood, anhedonia (decreased ability to experience happiness for natural rewards), irritability, difficulty concentrating, abnormal appetite or sleep, etc. The lifetime prevalence of depression is as high as 15%, and 15% of patients with severe depression may die by suicide. The 2005 Asian Mental Illness Summit Report showed that the number of depression patients in China has reached 26 million. According to statistics, the economic burden caused by depression worldwide accounts for about 4.4% of the total disease burden, which is comparable to ischemic heart disease or diarrheal diseases.

Recent studies have shown that changes in intestinal bacteria can affect the activity of the central nervous system of animals, thereby causing changes in brain function and behavior. The experimental results support the hypothesis of the “gut-brain axis”. However, the molecular mechanism and chemical basis of communication between the host and bacteria are still unclear. Gut bacteria may regulate brain function through the production of metabolites by microorganisms. These metabolites affect the biosynthesis of neurotransmitters, such as 5-HT (5-hydroxytryptamine), and thus affect the physiological function of the host. Therefore, clarifying the mode of action between the gut-brain pathway may help us understand the new functions of intestinal bacteria and help discover new methods for treating central nervous system disorders.

Morinda officinalis is a representative Chinese medicinal material of “Southern Medicine” in traditional Chinese medicine. It is the dried root of Morinda officinalis How, a plant of the Rubiaceae family. It has the effects of tonifying kidney and supporting Yang, strengthening tendons and bones, dispelling wind and eliminating dampness. It is used for impotence and spermatorrhea, cold pain in the lower abdomen, infertility due to uterine cold, irregular menstruation, rheumatic arthralgia, weakness of tendons and bones, etc. Morinda officinalis has complex chemical components and pharmacological effects. Its main components include sugars, anthraquinones, iridoid ether terpenoid glycosides, organic acids, trace elements, amino acids, sterols, etc.

The inventors found that the oral administration of Morinda officinalis oligosaccharides, especially purified pentasaccharides, could increase the concentration of 5-HT in the brain of depression model mice. Moreover, Morinda officinalis oligosaccharides could effectively increase the levels of 5-HTP (5-hydroxytryptophan) and 5-HT under the action of Clostridium butyricum. 5-HT is one of the most important endogenous neurotransmitters in humans and has a regulatory effect on depression. At present, there is a lack of safe and effective drugs with low toxic side effects for the treatment of depression in clinical practice.

### Contents of the Invention

In order to effectively solve the above problems, the present application provides a new class of drugs (Morinda officinalis oligosaccharides and their pentasaccharides) for preventing and/or treating depression, and the active ingredient, Morinda officinalis pentasaccharides, is separated and purified from Morinda officinalis oligosaccharides.

In depression model mice established by chronic moderate intensity unpredictable stimulation, it was found that after the oral administration of Morinda officinalis oligosaccharides, the rats' preference for sugar water increased significantly. However, after rats took Morinda officinalis oligosaccharides and antibiotics at the same time, their preference for sugar water did not change significantly, revealing that the intestinal bacteria of rats may play a role in preventing and/or treating depression under the action of Morinda officinalis oligosaccharides. In the forced swimming test, the model mice taking Morinda officinalis oligosaccharides or Morinda officinalis pentasaccharides showed improved and shortened passive floating time; and in the tail suspension test, the model mice taking Morinda officinalis oligosaccharides or Morinda officinalis pentasaccharides showed effectively shortened immobility time. The forced swimming test and the tail suspension test showed that Morinda officinalis oligosaccharides and pentasaccharides thereof could effectively prevent and/or treat depression, providing a scientific theoretical basis for the prevention or treatment of depression in clinical practice.

Therefore, in one aspect, the present invention relates to a use of Morinda officinalis oligosaccharides as shown in the structural formula or any combination thereof in the manufacture of a medicament for preventing and/or treating depression,

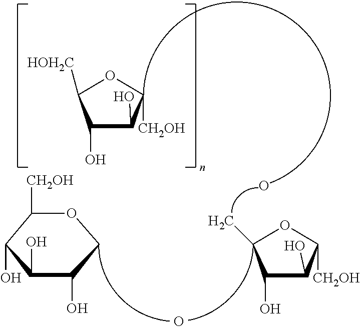

n=1, 2, 3, 4, 5, 6 or 7.

In some embodiments, in the structural formula, n=3.

In some embodiments, the depression is caused by a decrease in 5-hydroxytryptamine in the brain.

In another aspect, the present invention relates to a use of Morinda officinalis oligosaccharides as shown in the structural formula or any combination thereof in the manufacture of a medicament for increasing the level of 5-hydroxytryptophan and/or 5-hydroxytryptamine in a subject,

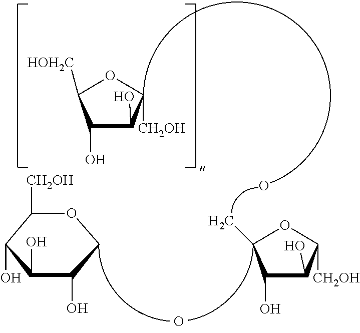

n=1, 2, 3, 4, 5, 6 or 7.

In some embodiments, in the structural formula, n=3.

In some embodiments, the medicament is used for increasing the level of 5-hydroxytryptophan and/or 5-hydroxytryptamine in the brain of the subject.

In some embodiments, the medicament is used for upregulating the level of peripheral 5-hydroxytryptophan and downregulating the level of 5-hydroxytryptamine, and increasing the level of 5-hydroxytryptamine in central nerve system.

In another aspect, the present invention relates to a use of Morinda officinalis oligosaccharides as shown in the structural formula or any combination thereof in the manufacture of a medicament for increasing the level of TPH and coenzyme BH₄ thereof in a subject, and/or decreasing the level of 5-HTPDC and coenzyme VB₆ thereof in a subject,

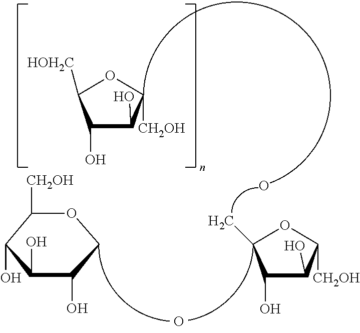

n=1, 2, 3, 4, 5, 6 or 7.

In some embodiments, in the structural formula, n=3.

Studies have found that the oral administration of Morinda officinalis oligosaccharides and purified pentasaccharides could increase the expression of 5-HT in the brain of depression model mice through intestinal flora, suggesting that Morinda officinalis oligosaccharides and purified pentasaccharides combined with beneficial intestinal flora could have the effect of preventing and/or treating depression, and that under the action of Clostridium butyricum, Morinda officinalis oligosaccharides could effectively increase the levels of 5-HTP and 5-HT. In particular, for patients with potential depression, the oral administration of Morinda officinalis oligosaccharides or purified monomers, preferably pentasaccharides, combined with intestinal flora could be used to regulate the concentration of 5-hydroxytryptamine (5-HT) in the brain, thereby achieving the effect of preventing depression.

Therefore, the medicament of the present invention further comprises an intestinal flora.

In some embodiments, the intestinal flora is selected from the genus Clostridium, such as Clostridium butyricum.

In some embodiments, the Morinda officinalis oligosaccharides or any combination thereof is in the same or different preparation units as the intestinal flora.

In another aspect, the present invention relates to a use of a composition in the manufacture of a medicament for preventing and/or treating depression, wherein the composition comprises one or more kinds of the Morinda officinalis oligosaccharides as shown in the structural formula and an intestinal flora,

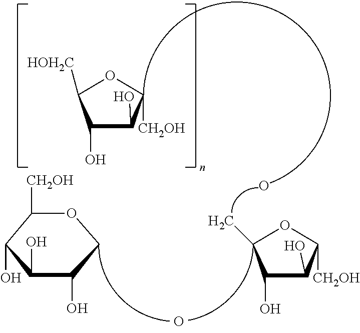

n=1, 2, 3, 4, 5, 6 or 7.

In some embodiments, the composition is composed of the Morinda officinalis oligosaccharides as shown in the structural formula and the intestinal flora, wherein, in the structural formula, n=3.

In some embodiments, the intestinal flora is selected from the genus Clostridium, such as Clostridium butyricum.

In some embodiments, the depression is caused by a decrease in 5-hydroxytryptamine in the brain.

In another aspect, the present invention relates to a use of a composition in the manufacture of a medicament for increasing the level of 5-hydroxytryptophan and/or 5-hydroxytryptamine in a subject, wherein the composition comprises one or more kinds of the Morinda officinalis oligosaccharides as shown in the structural formula and an intestinal flora,

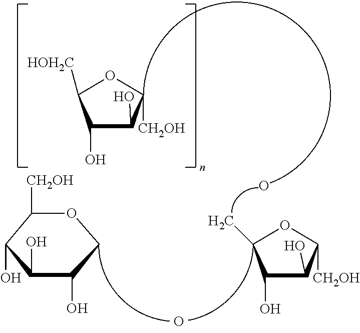

n=1, 2, 3, 4, 5, 6 or 7.

In some embodiments, the composition is composed of Morinda officinalis oligosaccharides as shown in the structural formula and the intestinal flora, wherein, in the structural formula, n=3.

In some embodiments, the intestinal flora is selected from the genus Clostridium, such as Clostridium butyricum.

In some embodiments, the medicament is used for increasing the level of 5-hydroxytryptophan and/or 5-hydroxytryptamine in the brain of the subject.

In some embodiments, the medicament is used for upregulating the level of peripheral 5-hydroxytryptophan and downregulating the level of 5-hydroxytryptamine, and increasing the level of 5-hydroxytryptamine in the central nerve system.

In another aspect, the present invention relates to a use of a composition in the manufacture of a medicament for increasing the level of TPH and coenzyme BH₄ thereof in a subject, and/or reducing the level of 5-HTPDC and coenzyme VB₆ thereof in a subject, wherein the composition comprises one or more kinds of Morinda officinalis oligosaccharides as shown in the structural formula and an intestinal flora,

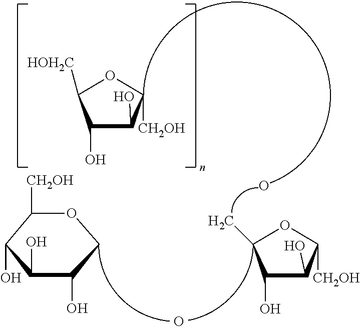

n=1, 2, 3, 4, 5, 6 or 7.

In some embodiments, in the structural formula, n=3.

In some embodiments, the intestinal flora is selected from the genus Clostridium, such as Clostridium butyricum.

In one aspect, the present invention provides a natural medicine Morinda officinalis oligosaccharides and monosaccharides thereof, preferably pentasaccharide, which can prevent and/or treat depression, and is used alone or in combination with an intestinal flora. It is revealed that the Morinda officinalis pentasaccharide component may be the main component of Morinda officinalis oligosaccharides for preventing and/or treating depression. Under the action of the intestinal flora, Morinda officinalis oligosaccharides, preferably pentasaccharide, increase the 5-HTP content in the blood through the Trp (tryptophan)-5-HTP (5-hydroxytryptophan)-5-HT (5-hydroxytryptamine) pathway. 5-HTP can pass through the blood-brain barrier and be converted into 5-hydroxytryptamine under the action of enzymes, thereby increasing the content of 5-HTP and/or 5-HT in the brain, and effectively preventing and treating depression through 5-hydroxytryptamine.

In the second aspect, the present invention also provides a relevant indicator (e.g., 5-hydroxytryptamine, 5-HT) for monitoring and evaluating depression.

The present invention includes but is not limited to detecting 5-HT in the feces, plasma and brain of each group of rats, and the detection method is mainly through LC-MS/MS.

In the third aspect, the present invention reveals that Morinda officinalis oligosaccharides can effectively promote the ratio of 5-hydroxytryptophan to tryptophan, that is, 5-HTP/Trp, under the action of intestinal bacteria.

In the fourth aspect, the present invention also reveals that tryptophan hydroxylase (TPH) plays a positive feedback regulatory role in the process of Morinda officinalis oligosaccharides regulating 5-HTP/Trp.

In the fifth aspect, the present invention reveals that Morinda officinalis oligosaccharides can effectively reduce the ratio of 5-hydroxytryptamine to 5-hydroxytryptophan, that is, the ratio of 5-HT/5-HTP, under the action of intestinal bacteria.

In the sixth aspect, the present invention also reveals that 5-hydroxytryptophan decarboxylase (5-HTPDC) plays a positive feedback regulatory role in the process of Morinda officinalis oligosaccharides regulating 5-HT/5-HTP.

In the seventh aspect, the present invention reveals that under the action of intestinal bacteria in vitro, a high dose (100 g/mL) of Morinda officinalis oligosaccharides can not only increase the levels of TPH enzyme and coenzyme tetrahydrobiopterin (BH₄) thereof, but also downregulate the levels of 5-HTPDC enzyme and its coenzyme vitamin B6 (VB₆) thereof.

In the eighth aspect, the present invention also discloses that rat brain homogenate can not only increase the levels of TPH and coenzyme BH₄ thereof, but also downregulate the levels of 5-HTPDC and coenzyme VB₆ thereof under the stimulation of a high dose (100 g/mL) of Morinda officinalis oligosaccharides.

In the ninth aspect, the present invention also separates and purifies the components such as trisaccharide, tetrasaccharide, pentasaccharide, hexasaccharide, heptasaccharide, octasaccharide and nonasaccharide in Morinda officinalis oligosaccharides.

In the tenth aspect, the present invention discloses that the pentasaccharide in Morinda officinalis oligosaccharides can not only increase the level of TPH, but also significantly downregulate the level of 5-HTPDC.

In the eleventh aspect, the present invention also discloses that the pentasaccharide in Morinda officinalis oligosaccharides is the main component for preventing and/or treating depression.

In the twelfth aspect, the present invention provides an intestinal bacterium that can be combined with Morinda officinalis oligosaccharides to prevent and/or treat depression.

In the thirteenth aspect, the intestinal bacterium involved in the present invention includes but is not limited to Clostridium butyricum.

In the fourteenth aspect, the present invention discloses that Morinda officinalis oligosaccharides effectively promote the level of 5-HTP under the action of Clostridium butyricum.

In the fifteenth aspect, the present invention also reveals that Morinda officinalis oligosaccharides can significantly increase the level of 5-HT under the action of Clostridium butyricum.

Compared with the prior art, the technical solution of the present invention has the following advantages:

First, it is revealed for the first time that Morinda officinalis oligosaccharides increase the 5-HTP content in the blood and the 5-HTP and 5-HT contents in the brain through the Trp-5-HTP-5-HT pathway under the action of intestinal flora, effectively improving the treatment of depression.

Second, the present invention also separates and purifies pentasaccharide from Morinda officinalis oligosaccharides and reveals that it plays an important role in preventing and/or treating depression.

Third, the present invention reveals that Morinda officinalis oligosaccharides can significantly increase the levels of 5-HTP and 5-HT under the action of Clostridium butyricum, and it is expected to provide a new solution for the prevention and/or treatment of depression.

### BRIEF DESCRIPTION OF THE DRAWINGS

The drawings described herein are used to provide a further understanding of the present invention and constitute a part of the present application. The schematic examples of the present invention and their descriptions are used to explain the present invention and do not constitute an improper limitation of the present invention. In the drawings:

FIG. 1 shows the structure of Morinda officinalis oligosaccharides.

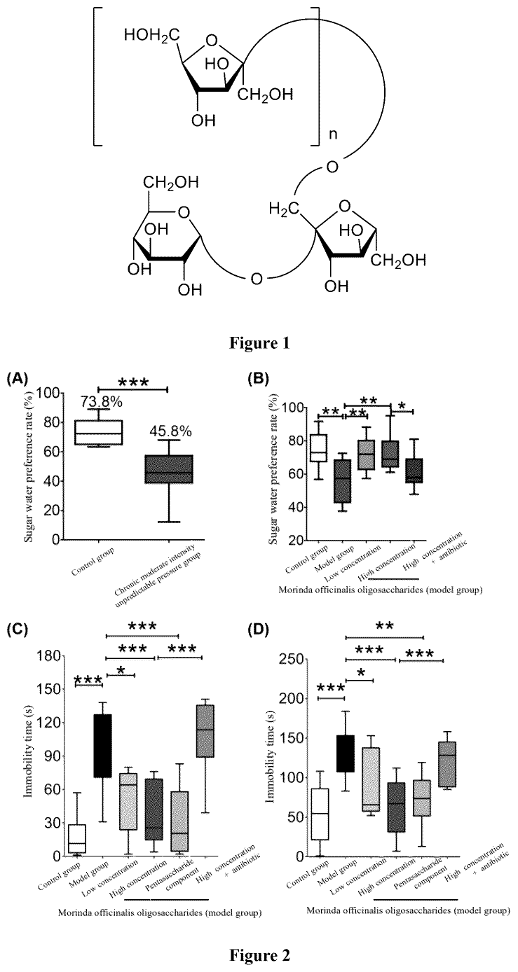

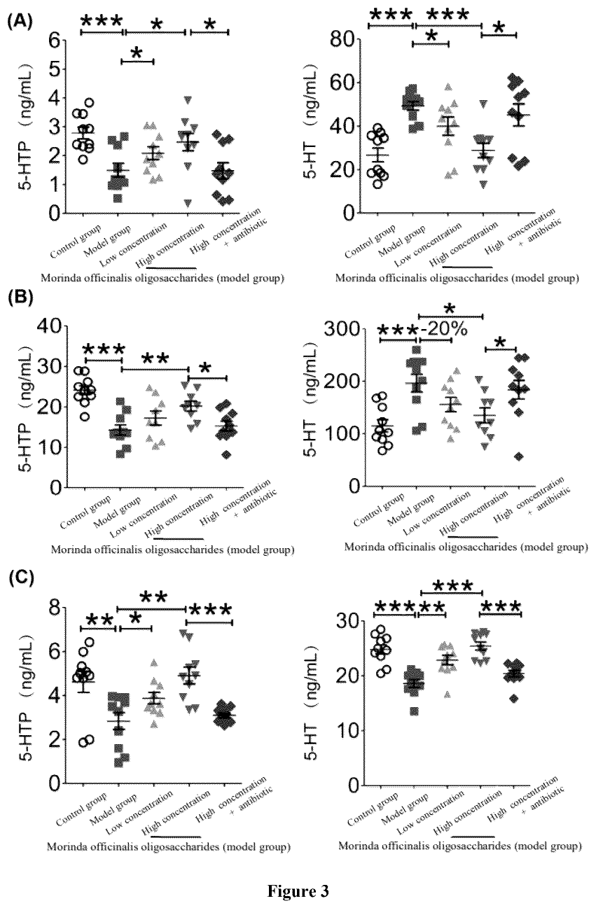

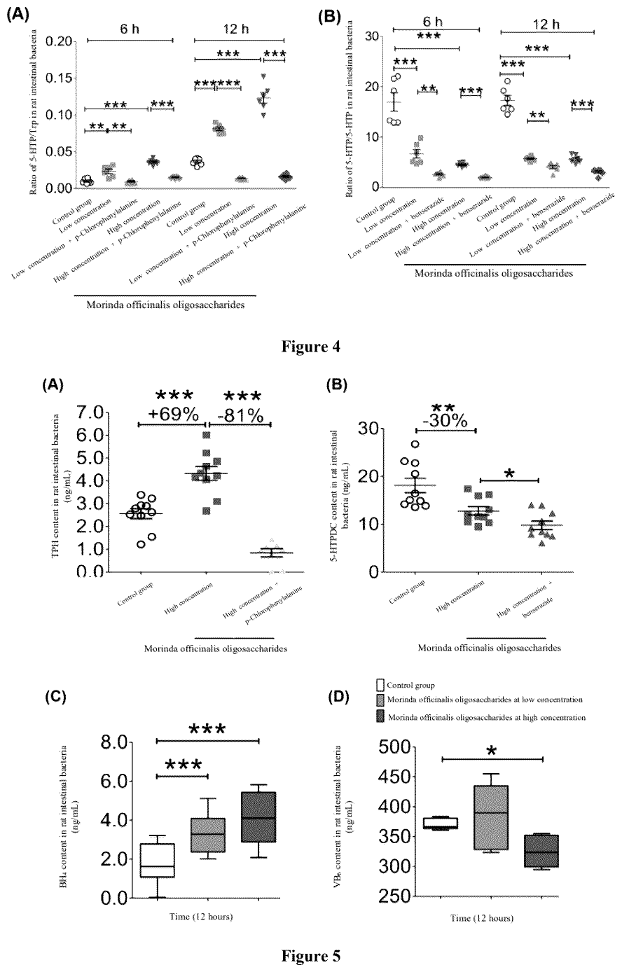

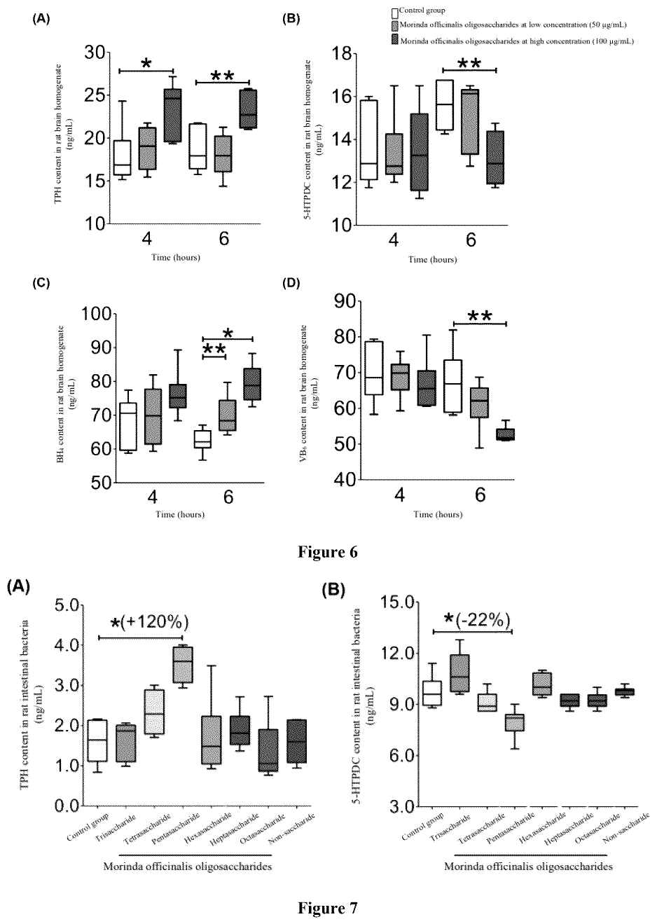

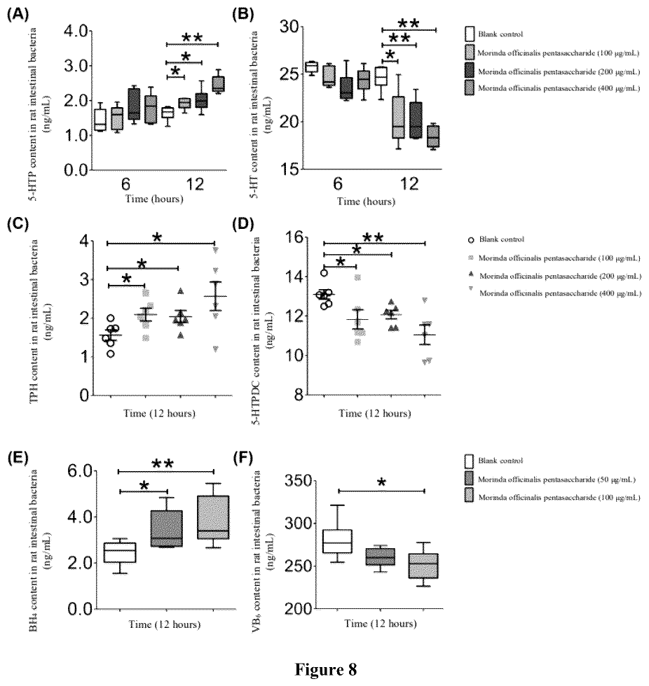

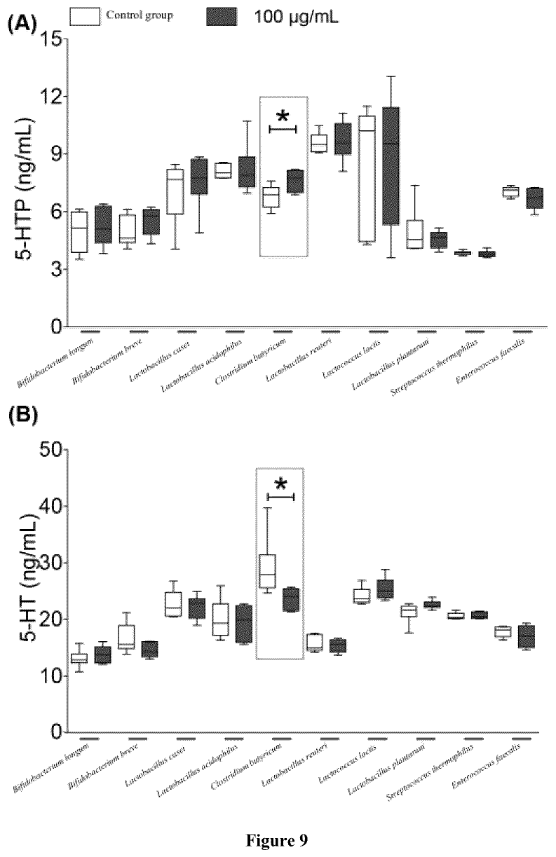

FIG. 2 shows the therapeutic effects of Morinda officinalis oligosaccharides and pentasaccharide thereof on depression model rats:
(A): construction of rat depression model;
(B): sugar water preference test of depression model rats after treatment with Morinda officinalis oligosaccharides;
(C): immobility time of depression model rats after treatment with Morinda officinalis oligosaccharides and pentasaccharide thereof, tested by pressure swimming;
(D): immobility time of depression model rats after treatment with Morinda officinalis oligosaccharides and pentasaccharide thereof, tested by tail suspension test.

FIG. 3 shows the contents of 5-HTP and 5-HT in rat feces (A), blood (B) and brain tissue (C) measured after depression model rats were treated with Morinda officinalis oligosaccharides.

FIG. 4 shows that under the action of intestinal bacteria, a high dose (100 g/mL) of Morinda officinalis oligosaccharides could not only regulate the ratio of 5-HTP/Trp (A), but also regulate the ratio of 5-HT/5-HTP (B).

FIG. 5 shows that under the action of intestinal bacteria, a high dose (100 g/mL) of Morinda officinalis oligosaccharides could not only regulate the levels of TPH enzyme (A) and coenzyme BH₄ thereof (C), but also regulate the levels of 5-HTPDC enzyme (B) and coenzyme VB₆ thereof (D).

FIG. 6 shows that in the rat brain homogenate, a high dose (100 g/mL) of Morinda officinalis oligosaccharides could not only regulate the levels of TPH enzyme (A) and coenzyme BH₄ thereof (C), but also regulate the levels of 5-HTPDC enzyme (B) and coenzyme VB₆ thereof (D).

FIG. 7 shows that under the action of intestinal bacteria, TPH (A) and 5-HTPDC enzyme (B) were regulated by various saccharide sugar components in Morinda officinalis oligosaccharides.

FIG. 8 shows that under the action of intestinal flora, Morinda officinalis pentasaccharides could not only regulate the levels of 5-HTP (A) and 5-HT (B), but also regulate the levels of TPH enzyme (C) and coenzyme BH₄ thereof (E); at the same time, it could also regulate the levels of 5-HTPDC enzyme (D) and coenzyme VB₆ thereof (F).

FIG. 9 shows that Morinda officinalis oligosaccharide combined with intestinal flora regulated 5-HTP (A) and 5-HT levels (B).

Unless otherwise specified, *p<0.05, **p<0.01, ***p<0.001.

### SPECIFIC MODELS FOR CARRYING OUT THE INVENTION

The technical solutions in the examples of the present invention will be clearly and completely described below in conjunction with the drawings in the examples of the present invention. Obviously, the described examples are only part of the examples of the present invention, not all of them. The following description of at least one exemplary example is actually only illustrative and is by no means a limitation on the present invention and its application or use. Based on the examples of the present invention, all other examples obtained by ordinary technicians in the field without creative work are within the scope of protection of the present invention.

### Example 1. Therapeutic Effect of Morinda officinalis Oligosaccharides in Depression Model Rats Established Under Chronic Moderate Intensity Unpredictable Stimulation

Sugar water intake and brain 5-HT concentration are one of the important indicators for evaluating the therapeutic effect of depression.

### 1. Experimental Animals, Instruments and Reagents

SD rats (male, 150±20 g) were purchased from Beijing Vital River Laboratory Animal Technology Co., Ltd., kept in an SPF environment (21±2° C., 12-hours light cycle) with free access to food and water during the experiment. Morinda officinalis oligosaccharides were purchased from Beijing Tongrentang Pharmacy. 5-HT standard was purchased from Beijing Solarbio Technology Co., Ltd.

### 2. Experimental Instruments and Analytical Methods

High-performance liquid chromatography-triple quadrupole tandem mass spectrometry (LC-MS/MS 8050, Shimadzu Corporation, Japan) was used to quantitatively determine 5-HT. In the experiment, Alltima C₁₈ (5 m, 4.6×150 mm) chromatographic column was used, and the column temperature was maintained at 40° C. Alltima C₁₈ (5 m, 4.6×150 mm) chromatographic column was used, and the column temperature was 40° C. The mobile phase contained water-formic acid (100:0.1 v/v) and methanol. Gradient elution (A:B, 0 min, 90:10; 1 min, 90:10; 1.01 min, 60:40; 5 min, 5:95; 7 min, 5:95; 7.01 min 90:10; 10 min 90:10) was used with a flow rate of 0.8 mL/min. Multiple reaction monitoring (MRM) mode was used for quantification, and the quantitative ion pair was 177.1>160.2.

### 3. Experimental Design and Animal Grouping

Experimental design: The experimental animals were divided into 5 groups, including normal group, model control group, model low-dose oral treatment group, model high-dose oral treatment group, and model pseudo-sterile oral treatment group. The model was established for 8 weeks and then treated for 14 days.

Animal grouping:
(1) Control group: No stimulation was performed, and an equal amount of normal saline was administrated orally during treatment.
(2) Model group: Stimulation was performed, and an equal amount of normal saline was administrated orally during treatment.
(3) Model low-dose treatment group: Stimulation was performed, and Morinda officinalis oligosaccharides (50 mg/kg/day) were administrated orally during treatment.
(4) Model high-dose treatment group: Stimulation was performed, and Morinda officinalis oligosaccharides (100 mg/kg/day) were administrated orally during treatment.
(5) Pseudo-sterile treatment group: Stimulation was performed, and Morinda officinalis oligosaccharides (100 mg/kg/day) and antibiotics were administrated orally during treatment; the doses of antibiotics were: cefadroxil (100 mg/kg/day)+oxytetracycline (300 mg/kg/day)+erythromycin (300 mg/kg/day).

### 4. Experimental Results

As shown in FIG. 2, 8 weeks after modeling, the sugar water intake of depression model rats was significantly lower than that of the normal rats, indicating that the model was successfully established. After 7 days of oral administration with Morinda officinalis oligosaccharides, the sugar water preference of model mice increased significantly, and the results of the two groups of 50 and 100 mg/kg/day of Morinda officinalis oligosaccharides showed dose-dependency. There was no significant change in the sugar water preference of the pseudo-sterile treatment group (oral administration of Morinda officinalis oligosaccharides and antibiotics at the same time), indicating that the oral administration of Morinda officinalis oligosaccharides was effective, which may be attributed to the effect produced by the stimulation of intestinal bacteria by Morinda officinalis oligosaccharides.

As shown in FIG. 3, the 5-HTP in the brain of the model group was significantly lower than that of the normal control group, indicating that the model was successfully established. Similarly, in the depression model rats after oral administration of Morinda officinalis oligosaccharides, the levels of 5-HTP and 5HT in the feces and blood of the rats were detected. The results confirmed that Morinda officinalis oligosaccharides at a high dose (100 μg/mL) could not only significantly increase the level of 5-HTP, but also reduce the level of 5-HT. In addition, Morinda officinalis oligosaccharides at a high dose could also effectively promote the levels of 5-HTP and 5-HT in brain tissue.

### Example 2. Effects of Morinda officinalis Oligosaccharides and Pentasaccharide Thereof on 5-HTP and 5-HT Under the Action of Intestinal Flora

5-HT synthesized by intestinal bacteria might be related to 5-HT in the brain.

### 1. Experimental Animals, Instruments and Reagents

SD rats (male, 200±20 g) were purchased from Beijing Vital River Laboratory Animal Technology Co., Ltd. The animals were kept in SPF environment (21±2° C., 12-hours light cycle) and allowed free access to food and water during the experiment. Morinda officinalis oligosaccharides and separated trisaccharides to heptasaccharides were provided by the Institute of Toxicology and Pharmacology, Academy of Military Medical Sciences, and 5-HT was purchased from Beijing Solarbio Technology Co., Ltd.

### 2. Experimental Design

Experimental design: Single sugar intestinal bacteria samples were divided into 3 groups, including control group, 50 g/mL and 100 g/mL groups, and two time points of 6 hours and 12 hours;

SD rat intestinal bacteria samples were divided into 3 groups, including control group, 50 μg/mL and 100 g/mL groups, and two time points of 6 hours and 12 hours.

SD rat brain homogenate samples were divided into 3 groups, including control group, 50 μg/mL and 100 g/mL groups, and two time points of 4 hours and 6 hours, in the in vitro model.

SD rat intestinal bacteria samples were divided into 4 groups, including control group, 100 μg/mL, 200 g/mL and 400 g/mL groups (or 50 g/mL, 100 g/mL), and two time points of 6 hours or 12 hours.

### 3. Experimental Results

As shown in FIG. 4, the addition of high dose (100 g/mL) of Morinda officinalis oligosaccharides to the intestinal bacteria of SD rats could not only effectively increase the ratio of 5-HTP/Trp, but also effectively reduce the ratio of 5-HT/5-HTP.

As shown in FIG. 5, the addition of high dose (100 g/mL) of Morinda officinalis oligosaccharides to the intestinal bacteria of SD rats could not only effectively promote the levels of TPH and coenzyme BH₄ thereof, but also effectively reduce the levels of 5-HTPDC and coenzyme VB₆ thereof.

As shown in FIG. 6, the addition of high dose (100 g/mL) of Morinda officinalis oligosaccharides to the brain homogenate of SD rats could not only effectively promote the levels of TPH and coenzyme BH₄ thereof, but also effectively reduce the levels of 5-HTPDC and coenzyme VB₆ thereof.

As shown in FIGS. 7 to 8, under the action of intestinal flora, the pentasaccharide component of Morinda officinalis could not only upregulate the levels of TPH and coenzyme BH₄ thereof, but also inhibit the levels of 5-HTPDC and coenzyme VB₆ thereof, at the same time, it could also upregulate 5-HTP and downregulate 5-HT, which made it have a good antidepressant effect.

### Example 3. Morinda officinalis Oligosaccharides Capable of Significantly Increasing Levels Of 5-HTP and 5-HT Under Action of Intestinal Bacteria (Clostridium butyricum)

Enterococcus faecalis, Bifidobacterium longum, Bifidobacterium breve, Lactobacillus casei, Lactobacillus acidophilus, Clostridium butyricum, Lactobacillus reuteri, Lactococcus lactis, Lactobacillus plantarum and Streptococcus thermophilus were purchased from Nanjing Bianzhen Biotechnology Co., Ltd. One sample of each of the 10 strains was taken out from the −80° C. freezer and quantified after activation in its corresponding culture system, and the number of single bacterial cells of each strain was controlled to ensure that the same number of cells in each strain was used. MOO solutions (5 mg/mL and 10 mg/mL) were prepared in water, and 5 μL of MOO solution was added to 500 μL of culture medium to give a final concentration of MOO of 50 μg/mL or 100 μg/mL. After culturing at 37° C. for 12 hours, 300 μL of acetonitrile precipitated protein was added to 100 μL of bacterial solution, and 5-HTP and 5-HT levels were analyzed by LC-MS/MS.

As shown in FIG. 9, Morinda officinalis oligosaccharides could effectively increase the levels of 5-HTP and 5-HT under the action of Clostridium butyricum.

In addition to those described herein, various modifications of the present invention will be obvious to those skilled in the art based on the foregoing description. Such modifications are also intended to fall within the scope of the appended claims.

1. A method of preventing and/or treating depression, comprising administrating Morinda officinalis oligosaccharides as shown in the structural formula or any combination thereof, 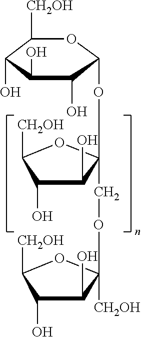
n=1, 2, 3, 4, 5, 6 or 7.

2. The method according to claim 1, further comprising administrating an intestinal flora.

3. The method according to claim 1, wherein, in the structural formula, n=3.

4. The method according to claim 2, wherein, in the structural formula, n=3.

5. The method according to claim 2, wherein the intestinal flora is selected from the genus Clostridium.

6. The method according to claim 1, wherein the depression is caused by a decrease in 5-hydroxytryptamine in brain.

7. A method, comprising administrating Morinda officinalis oligosaccharides as shown in the structural formula or any combination thereof, 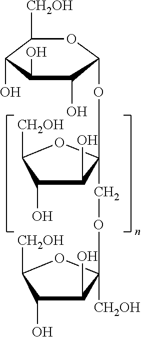
n=1, 2, 3, 4, 5, 6 or 7;
wherein the method is to increase the level of 5-hydroxytryptophan and/or 5-hydroxytryptamine: upregulate the level of peripheral 5-hydroxytryptophan and downregulate the level of 5-hydroxytryptamine, and increase the level of central 5-hydroxytryptamine; or increase the level of TPH and coenzyme BH₄ thereof and/or reduce the level of 5-HTPDC and coenzyme VB₆ thereof in a subject.

8. The method according to claim 7, further comprising administrating an intestinal flora.

9. The method according to claim 7, wherein, in the structural formula, n=3.

10. The method according to claim 8, wherein, in the structural formula, n=3.

11. The method according to claim 8, wherein the intestinal flora is selected from genus Clostridium.

12. The method according to claim 7, wherein the method is to increase the level of 5-hydroxytryptophan and/or 5-hydroxytryptamine in the brain of the subject.

13. (canceled)

14. (canceled)

15. (canceled)

16. The method according to claim 2
wherein the Morinda officinalis oligosaccharides or any combination thereof and the intestinal flora are in the same or different preparation units.

17. (canceled)

18. The method according to claim 5, wherein the intestinal flora is Clostridium butyricum.

19. The method according to claim 11, wherein the intestinal flora is Clostridium butyricum.

20. The method according to claim 8, wherein the Morinda officinalis oligosaccharides or any combination thereof and the intestinal flora are in the same or different preparation units.
